(19) United States
(12) Patent Application Publication
(10) Pub. No.: US 2023/0405075 A1
(43) Pub. Date: Dec. 21, 2023

(54) PREPARATIONS CONTAINING MITRAGYNE EXTRACT OR ITS ISOLATED ALKALOIDS AND CANNABIS EXTRACT OR ITS ISOLATED CANNABINOIDS, AND COSMETIC AND/OR PHARMACEUTICAL USE THEREOF

(71) Applicant: HERBA INVEST, PTE. LTD., Singapore (SG)

(72) Inventor: Giancarlo CRAVOTTO, Torino (IT)

(21) Appl. No.: 18/024,788

(22) PCT Filed: Jul. 8, 2020
(86) PCT No.: PCT/IB2020/056400

§ 371 Date: Mar. 6, 2023

(51) Int. Cl. A61K 36/74; A61K 9/06; A61K 36/185; A61K 31/00; A61P 29/00; A61P 17/06; A61K 9/00; A61K 31/4375

(52) U.S. Cl. CPC A61K 36/74; A61K 9/06; A61K 36/185; A61K 31/658; A61P 29/00; A61P 17/06; A61K 9/007; A61K 31/4375

(57) ABSTRACT

A cosmetic and pharmaceutical preparation for topical use contains from about 0.00001 wt.-% to about 20 wt.-%, of one or more alkaloids of Mitragyna speciosa and from about 0.00001 wt.-% to about 20 wt.-% of one or more cannabinoids of Cannabis indica or Cannabis sativa. The preparation is intended for use in preventing and/or treating skin and mucosal disorders and conditions.

### TECHNICAL FIELD

The present invention relates to preparations containing active principles derived from mytragyne (in particular Mitragyna speciosa) and cannabis (in particular Cannabis sativa or Cannabis indica), and more particularly to preparations containing Mitragyna speciosa extract or its isolated alkaloids and Cannabis extract or its isolated cannabinoids. The invention also relates to cosmetic and/or pharmaceutical use of such preparations, preferably topic use and inhalatory use.

### BACKGROUND ART

There is a growing interest in developing pharmaceutical preparations making use of natural active principles, as a replacement of synthetic drugs for the treatment of several diseases and/or disorders.

As far as skin conditions and/or disorders are concerned, use of preparations based on active principles derived from Mitragyna speciosa or Cannabis (hemp) is already known.

Mitragyna speciosa (also known as Kratom) is a tree belonging to the Rubiaceae family, indigenous to Southeast Asia, Indonesia and Papua New Guinea. Its leaves are known to possess stimulant and opioid-like analgesic effects (at low and high dosages, respectively), and actually they have found widespread use for hundreds of years, generally chewed or made into a tea-like infusion. A high number of pharmacological active alkaloids have been individuated in kratom, the principal ones including mitragynine (MG), 7-hydroxymitragynine (HMG), speciociliatine (SC), speciogynine (SG) and paynantheine (P). Most of the opioid-like activity of kratom has been attributed to MG and HMG, both of which exhibit dose-related antinociceptive effects and stimulate μ- and δ-opioid receptors. The concentrated extract also contains several polyphenols with strong antioxidant activity and antimicrobial properties.

The opioid-like activity of kratom has been since years exploited for pharmaceutical compositions, mainly directed to treatment of pain. Reference can be made for instance to U.S. Pat. Nos. 3,324,111, 3,256,149 and WO2017165738. US20150352100 includes Mitragyna speciosa among several active compounds and plants to be used as selective delta-opioid receptor antagonists and specific sensory receptor ligands for treatment of skin diseases and conditions, including skin wounds, skin aging and skin tumours, and for improving skin repair. The preparations of US20150352100 are intended for oral or topical administration.

In turn Cannabis is a genus of flowering plants of the Cannabaceae family, indigenous to Central Asia. Cannabis contains psychoactive constituents (cannabinoids), the main of which are Δ(9)-tetrahydrocannabinol (THC), cannabidiol (CBD), tetrahydrocannabinolic acid (THCA) and cannabidiolic acid (CBDA). Due to its contents in psychoactive substance, Cannabis is scheduled by many national regulations and by international conventions, which restrict the access to potential therapies and cosmetic applications, even if in 2018 the WHO (World Health Organization) has proposed rescheduling Cannabis within international law to take account of the growing evidence for medical applications of the plant [WHO 2018]. Among cannabinoids, THC (which is principal psychoactive agent) is scheduled, so that there is a growing interest in Cannabis strains containing less than 0.3% THC by dry weight. By international convention and legal definition, those strains are named “hemp”, the usual name of the plant when used for industrial applications other than the medical/pharmaceutical ones.

Numerous publications and patent applications have demonstrated anti-inflammatory and anti-pruritic properties and have highlighted a potential therapeutic role of cannabinoids in the management of atopic dermatitis and allergic contact dermatitis and other skin diseases and/or disorders. Reference can be made for instance to the paper by Challapalli et al. 2002 and to Patent Application publications WO 2019/191830 A1, WO 2019/144190 A1, WO 2019/144189 A1, WO 2019/140357 A1, WO 2019/130215 A1 and WO 2019/056123.

Also preparations based on Cannabis derivatives other than cannabinoids, in particular a cream with 3% seed extract (Cannabis seeds do not contain cannabinoids) showed the efficacy in the reduction of human cheek skin sebum and erythema content (Ali 2015)

Hitherto however, treatment effectiveness by means of compositions containing opioid-like alkaloids of Mitragyna speciosa or cannabinoids of Cannabis sativa has been achieved only with relatively high contents of the active principles. This is a serious problem, since in particular the oral use of preparations containing extracts of Mitragyna speciosa is associated with serious side effects such as addiction and liver damage, and high amounts of cannabinoids show similar side effects, in particular on cognitive performances.

Taking this into account, the inventors have performed studies directed at finding preparations for treatment of skin disorders, characterized by potentiated pharmacological properties and negligible negative side effects compared to the state of the art.

Surprisingly, it has been found that this problem can be solved by cosmetic and dermatological preparations containing a combination of extracts of Mitragyna speciosa and extracts of Cannabis sativa or C. indica. The combination shows complementary and even synergistic effects regarding treating skin disorders including inflammation, acne, atopic dermatitis, psoriasis, pruritus and sub-cutaneous pain as well as an anti-ageing effect. The same effects of the combined extracts were observed on mucosae (mucous membranes) of the mouth and genitals with a remarkable reduction of inflammation, pain, swelling (oedema). Worthy of note are the beneficial effects on mucous membrane pemphigoid and on hemorrhoids at low concentrations (0.1 to 0.5% of both purified extracts). This leads to minimisation of negative side effects as lower concentrations of the active principles derived from Mitragyna speciosa and Cannabis are required with respect to compositions containing Mitragyna alkaloids or Cannabis cannabinoids alone. At high concentrations the synergistic effect is no longer detectable, either for the enhanced skin enrichment and transdermal permeation, or for the improved pharmacological effects.

Compositions containing both alkaloids of Mitragyna speciosa and cannabinoids of Cannabis sativa are disclosed in US 2018/193399 A1. The alkaloids and cannabinoids can be present as extracts from the respective plant or can be included as synthesised compounds. The compositions are intended for treating spasms, inflammation and pain associated with cancer, trauma, medical procedure and neurological diseases and disorders, and the document states that the combination of both extracts is more effective than each extract alone. According to the document, depending on the kind of disorder to be treated, the composition may be administered by different routes, including transdermal and other topical routes or inhalatory route. A problem with this prior art composition is that it uses high concentrations of active principles (cannabinoids such as THC, CBD and CBG (cannabigerol) amount up to 70% by mass of the total composition, and alkaloids such as HMG, MG, mitrafoline or mitraphylline amount up to 90% by mass of the total composition), what can give rises to serious side effects. The document does not specifically disclose use of the compositions for skin affections, nor teaches how to exploit synergic properties of both kinds of components.

### SUMMARY Of INVENTION

It is an object of the present invention to solve the problems mentioned above of the prior art, by providing a composition for pharmaceutical and/or cosmetic use, containing active principles derived from both Mitragyna speciosa and Cannabis sativa or Cannabis indica, which is more effective than the prior art compositions with lower contents of the active principles, whereby a reduction of the side effects can be achieved.

This problem is solved by a composition containing at most 20 wt.-%, preferably from about 0.00001 wt.-% to about 20 wt.-%, more preferably from about 0.0001 wt.-% to about 10 wt.-%, most preferably from about 0.1 wt.-% to about 5 wt.-% of one or more alkaloids from Mitragyna speciosa, and at most 20 wt.-%, preferably from about 0.00001 wt.-% to about 20 wt.-%, more preferably from about 0.0001 wt.-% to about 10 wt.-%, most preferably from about 0.1 wt.-% to about 5 wt.-% of one or more cannabinoids, the weight percentages being related to the total amount of the preparation.

Preferably, the alkaloids are selected from: speciociliatine (SC), 3-isopaynantheine (IP), speciogynine (SG), paynantheine (P) and mitragynine (MG), and the cannabinoids are selected from Δ(9)-tetrahydrocannabinol (THC), cannabidiol (CBD), tetrahydrocannabinolic acid (THCA) and cannabidiolic acid (CBDA).

In a preferred embodiment, the alkaloids and the cannabinoids are present as extracts, in particular alcoholic extracts, from leaves of Mitragyna speciosa and from inflorescences of Cannabis sativa or Cannabis indica, respectively. The amount of each extract is such that the preparation contains the preferred amounts indicated above of alkaloids and cannabinoids.

In another embodiment, the isolated alkaloids and cannabinoids are present in the composition.

The inventive preparations show significant complementary and synergistic effects especially in treatment of skin disorders and/or conditions and pain, as well in treatment of mucosal disorders. Skin disorders and/or conditions include, without being limited to, ageing and redness, inflammation, acne, atopic dermatitis, psoriasis, couperose, dandruff, scalp disorders, excessive sweating (hyperhidrosis), pruritus and sub-cutaneous pain. As regards mucosae, regenerating, lenitive, soothing and healing effects have been observed on several lip disorders, rhagades and haemorrhoids, where a remarkable reduction of inflammation, pain, swelling (oedema) was observed. Worthy of note are the beneficial effects on mucous membrane pemphigoid and on hemorrhoids at low concentrations (0.1 to 0.5% of both purified extracts), as said before.

A topical (transdermal or transmucosal) route for the administration of the inventive preparation is potentially ideal for localised symptoms, such as those found in the disorders mentioned above. Topical administration allows a steady infusion of both kinds of active principles over a prolonged period of time, while also minimising the adverse effects of higher drug peak concentrations, which can improve patient adherence, in dermatological conditions and in particular the anti-aging effect.

The combination of the Mitragyna alkaloids and the cannabinoids enables lower dosages for both purified extracts (or both kinds of active principles), with the advantage to get stronger biological activity minimizing any possible side effects or toxicity. Owing to the difference in polar moieties among Mitragyna alkaloids and cannabinoids, a selective transdermal permeation is known from the literature. According to a preferred embodiment of the invention, however, the very low permeation of more lipophilic mitragynine (MG) and tetrahydrocannabinol (THC) can be improved by means of suitable enhancers or ethosomal carriers.

We underline that, in case of both skin and mucosae, at high concentrations the synergistic effect is no longer detectable, either for the enhanced skin enrichment and transdermal permeation, or for the improved pharmacological effects.

A preparation according to the invention for topical application can have a dosage form selected from ointment, O/W-emulsion, W/O-emulsion, dispersion, lotion, gel, mousse, tincture, foam, powder, tape and patch.

A cosmetic, dermatological or pharmaceutical preparation according to the invention will contain pharmaceutically acceptable auxiliaries, additives and excipients customary for the dosage forms indicated above, e. g. surfactants, emulsifiers, cationic polymers, thickeners, film formers, antimicrobial active ingredients, astringents, antioxidants, UV light-screen filters, pigments/micropigments, gelling agents, and further additives customary in cosmetics, such as, for example, super fatting agents, moisturizing agents, silicones, stabilizers, conditioning agents, glycerol, preservatives, pearlizing agents, colorants, fragrance and perfume oils, solvents, hydrotropes, opacifiers, fatty alcohols, substances having keratolytic and keratoplastic action, antidandruff agents, biogenic active ingredients (local anaesthetics, antibiotics, anti-inflammatories, antiallergics, corticosteroids, sebostatics), vitamins, Bisabolol®, Allantoin®, Phytantriol®, Panthenol®, AHA acids (alpha-hydroxy acids), plant extracts, for example Aloe vera, and proteins.

The inventive preparations can potentially replace cortisone and other steroidal anti-inflammatory drugs, in particular in treatment of atopic dermatitis and eczema as well as of psoriasis. The advantages inherent in such a possibility are immediately apparent, taking into account that prolonged use of corticosteroids induces broadband immuno-suppression and may increase the risk of cancer. A further positive aspect in replacing corticosteroids by the inventive preparations is that the latter also reduce pain, and not only inflammation. It is also to be appreciated that the inventive preparations, as substitute for cortisone, are considerably more effective than compositions containing only alkaloids or only cannabinoids, as it will be discussed later on with reference to the Examples

A preparation according to the invention, with low concentrations of the active principles (e. g. <0.5% each), can also have a dosage form suitable for inhalatory application, in particular spray or aerosol. Such a dosage form is suitable for treating disorders and/or conditions of the respiratory tract, including, without being limited to, sinusitis, adenoiditis, laryngitis, nasopharyngitis, chronic rhinitis, chronic pharyngitis and tracheitis, bronchiolitis and bronchitis. Such a dosage form can also be used in combination with a dosage form for topical application in order to enhance the effects of the latter.

The transdermal and transmucosal route or the intranasal administration also obviates the low oral bioavailability of cannabinoids.

According to another aspect, the invention also concerns use of the preparations described above for treating skin and mucosal disorders and conditions and/or for treating disorders and/or conditions of the respiratory tract.

According to yet another aspect of the invention, the inventive synergistic effect at low concentrations of Mitragyna purified extract or isolated alkaloids and Cannabis purified extract or isolated cannabinoids (e.g. concentrations of each of the active principles lower than 0.5%) can be exploited in oral administration or any other systemic administration (e.g. aerosol) in order to potentiate the topical treatment. This combination at low concentrations can maximize the pharmacological effects and minimize the undesirable side effects.

### BRIEF DESCRIPTION OF DRAWINGS

The invention will now be further described with reference to the following nonlimiting examples and to the accompanying drawing.

FIG. 1 shows a comparison among the results of transcutaneous permeation tests performed on formulations according to the invention and on formulations including only Mitragyna alkaloids or only cannabinoids.

### EXAMPLES

### Example 1

A preparation according to the invention was formulated for topical use as a gel emulsion (oil/water, o/w, cream) having the following composition (percentages in weight):
Aqua (water): 60-80%
Carbomer: 0.1-0.3%;
Acrylates/C10-30 Alkyl Acrylate Cross-polymer: 0.1-0.2%;
Isopropyl Myristate: 10-15%;
Phenoxyethanol/Methylparaben/Buthylparaben/Ethylparaben/Propylparaben:
0.1-0.7%;
Mitragyna purified extract (or isolated alkaloids) alcoholic solution: 3-20% (EtOH); (alkaloids concentration in the final formulation from 0.1 to 5%);
Cannabis purified extract (or isolated cannabinoids) alcoholic solution: 2-15% (EtOH); (cannabinoids concentration in the final formulation from 0.1 to 5%).

1 N Sodium Hydroxide solution was added until the pH was about 6.0.

### Example 2

A preparation according to the invention was formulated for topical use as a water/oil (w/o) cream having the following composition (percentages in weight):
Diisostearoyl Polyglyceryl-3 Dimer Dilinoleate: 1.5-5%;
Synthetic Beeswax: 1-0.5%;
Hydrogenated Castor Oil: 0.1-0.8%;
Paraffinum liquidum: 2-9%;
Isopropyl Myristate: 2-9%;
Hydrogenated polyisobutene: 3-9%;
Aqua water: 55-75%;
Magnesium sulphate heptahydrate: 0.5-3%;
Mitragyna purified extract (or isolated alkaloids) alcoholic solution: 3-15% (EtOH); (alkaloids concentration in the final formulation from 0.1 to 5%);
Cannabis purified extract (or isolated cannabinoids) alcoholic solution: 2-15% (EtOH) (cannabinoids concentration in the final formulation from 0.1 to 5%).

The production of the inventive preparations can be carried out, for example, by adding together the individual components in turbo mixers or ultra-turrax for 40 sec, optionally with slight warming to about 50° C.

In the above examples, the extracts were obtained as follows:

1) Extraction of Mitragyna leaves with Ethanol (EtOH) under reflux.

Mitragyna leaves (450 g) were introduced in a round bottomed flask, EtOH was added (4.5 L) and the suspension was heated at reflux under magnetic stirring for 2 h. The suspension was filtered, and the liquid phase was dried under vacuum. The same procedure was repeated on residual leaves using fresh EtOH (4.5 L). After filtration, EtOH from the second extraction was added to the first sample, dried under vacuum, affording 120 g of a dark green powder (yield 27-29%).

2) Cannabis Extraction with Ethanol (EtOH) Under Reflux

Cannabis inflorescences (400 g) were introduced in a round bottomed flask, EtOH was added (4.5 L) and the suspension was heated at reflux under magnetic stirring for 2 h. The suspension was filtered, and the liquid phase was dried under vacuum affording 140 g of a brownish powder (yield 33-39%). Ca. 50% of cannabinoids were recovered in the active decarboxylated form. One additional hour heating under light vacuum at 120° C. leads to complete decarboxylation.

In an alternative method of preparation of the above formulations, extraction of Mitragyna leaves took place with 1:1 MeOH/H2O at room temperature, as follows:

50 g of plant material were placed into a round bottomed flask (1 L), 500 ml of a MeOH/H2O 1:1 mixture was added, and the suspension was kept under magnetic stirring at room temperature for 24 h. The suspension was filtered on paper in a Büchner funnel and MeOH was evaporated under vacuum. A 0.1 M NH4OH solution was added to the residual suspension to give a pH value of 9. The resulting suspension was extracted with CH2Cl2, which was evaporated to give the crude extract. The extract was dissolved in HCl 0.1 N (pH=3) and washed with petroleum ether (3 times). A 0.1 M NH4OH solution was added to the acidic solution to give a pH value of 9. The alkaloids precipitated. The precipitate was filtered on paper in a sintered glass filter, dried and weighed (yield 1.2-1.3%), while the residue on the filter and the basic solution were extracted with CH2Cl2.

### Example 3

A w/o cream similar to that of Example 2 was prepared, also including an enhancer for increasing transdermal permeation. The ethosomal system used as enhancer had the following composition:
Phospholipon 90: 1-3% w/w
ethanol: 3-20% w/w
ethanolic solution of alkaloids and cannabinoids
distilled water: ad 100% w/w.

Water was added in a fine stream under sonication at 30-35° C. throughout the preparation and was then left to cool at room temperature.

The resulting preparation had the following final formulation (percentages in weight):
Phospholipon 90: 1-3%
Diisostearoyl Polyglyceryl-3 Dimer Dilinoleate: 1.5-5%;
Hydrogenated Castor Oil: 0.1-0.8%;
Isopropyl Myristate: 2-8%;
Hydrogenated polyisobutene: 3-7%;

Aqua water: 50-70%;
Mitragyna purified extract (or isolated alkaloids) alcoholic solution: 3-15% (EtOH), (alkaloids concentration in the final formulation from 0.1 to 5%)
Cannabis purified extract (or isolated cannabinoids) alcoholic solution: 2-15% (EtOH), (cannabinoids concentration in the final formulation from 0.1 to 5%).

### Example 4 (Comparison Example)

A water/oil (w/o) cream containing only Mitragyna extract was prepared. The cream had the following composition (percentages in weight):
Diisostearoyl Polyglyceryl-3 Dimer Dilinoleate: 1.5-5%;
Synthetic Beeswax: 0.1-0.5%;
Hydrogenated Castor Oil: 0.1-0.8%;
Paraffinum liquidum: 2-9%;
Isopropyl Myristate: 2-9%;
Hydrogenated polyisobutene: 3-9%;
Aqua water: 55-75%;
Magnesium sulphate heptahydrate: 0.5-3%;
Mitragyna purified extract (or isolated alkaloids) alcoholic solution: 3-15% (EtOH); (alkaloids concentration in the final formulation from 0.1 to 5%);

### Example 5 (Comparison Example)

A water/oil (w/o) cream containing only Cannabis extract was prepared. The cream had the following composition (percentages in weight):
Diisostearoyl Polyglyceryl-3 Dimer Dilinoleate: 1.5-5%;
Synthetic Beeswax: 0.1-0.5%;
Hydrogenated Castor Oil: 0.1-0.8%;
Paraffinum liquidum: 2-9%;
Isopropyl Myristate: 2-9%;
Hydrogenated polyisobutene: 3-9%;
Aqua water: 55-75%;
Magnesium sulphate heptahydrate: 0.5-3%;
Cannabis purified extract (or isolated cannabinoids) alcoholic solution: 2-15% (EtOH); (cannabinoids concentration in the final formulation from 0.1 to 5%).

The Mitragyna and Cannabis extracts were prepared according to the methods described above in 1 and 2, respectively.

The transcutaneous permeation of w/o creams according to Examples 2 and 3 were evaluated by using the Franz cell diffusion assay and compared with the permeation of w/o creams containing Mitragyna alkaloids only and Cannabis cannabinoids only, according to examples 4 and 5.

As known, Franz cell diffusion assay is the most common and reliable method to evaluate the transcutaneous permeation of molecules. Such an assay is well known in the art and a detailed description is not necessary.

The assay conditions were as follows:
cells used: 0.9 and 1.4 mm diameter.
membrane: porcine ear skin, 1 mm thickness.
temperature of incubation: 32° C.

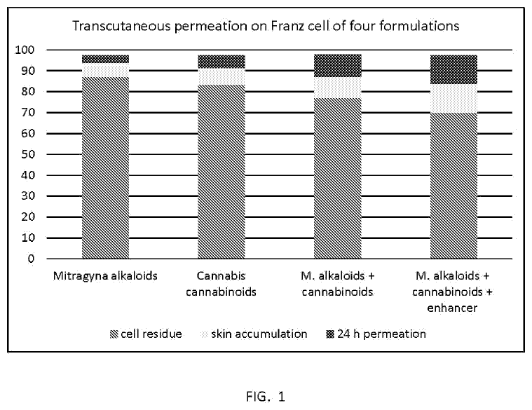

The results of the evaluations are shown in FIG. 1, where the different bars relate to:
a cream according to Example 4, with 1% Mitragyna alkaloids;
a cream according to Example 5, with 1% cannabinoids;
a cream according to Example 2, containing 0.5% purified Mitragyna alkaloids and 0.5% cannabinoids;
a cream with enhancer according to Example 3, also containing 0.5% purified Mitragyna alkaloids and 0.5% cannabinoids,
respectively.

The particular w/o cream according to Example 2 used in the evaluation of transcutaneous permeation had the following composition (percentages in weight):
Diisostearoyl Polyglyceryl-3 Dimer Dilinoleate: 3%
Synthetic Beeswax: 0.2%
Hydrogenated Castor Oil: 0.3%
Paraffinum liquidum: 7%
Isopropyl Myristate: 7%
Hydrogenated polyisobutene: 7.5%
Aqua (water): 60%
Magnesium sulphate heptahydrate: 1%
Mitragyna purified extract (or isolated alkaloids) alcoholic solution: the amount/concentration resulting in a 0.5% alkaloid concentration of in the final formulation;
Cannabis purified extract (or isolated cannabinoids) alcoholic solution: the amount/concentration resulting in a 0.5% alkaloid concentration in the final formulation.

As depicted in the diagram of FIG. 1, by using water/oil cream with 1% alkaloids or cannabinoids alone, the biggest fraction of Mitragyna alkaloids and the purified cannabinoids fraction from Cannabis remain in the epidermis. The percentage of skin accumulation summed to the permeation was 10.3% and 14.1% respectively. The combined formulation water/oil cream containing 0.5% Mitragyna alkaloids and 0.5% cannabinoids showed an overall increase percentage of skin accumulation and transdermal permeation up to 20.8%; the presence of enhancer blend resulted in a further increase, up to 27.6%, of the summed skin accumulation and transdermal permeation.

It is to be appreciated that the skin accumulation and even more the transdermal permeation in the presence of the combined extracts are however significantly increased in selective way. As regards the alkaloids, the highest concentrations detected in Franz cell receptor were, in order of selective passage: speciociliatine, isopaynantheine and, in lower percentage, mitragynine and paynantheine, whereas the other alkaloids could not be detected. As regards the cannabinoids, an increase in concentration in Franz cell receptor was observed for cannabidiol.

In order to demonstrate the synergistic effect of a preparation according to the invention, an in vivo evaluation of its anti-inflammatory and anti-psoriatic effects was performed. In both cases, the evaluations were performed by means of topical applications of a w/o cream.

A): In Vivo Evaluation of Anti-Inflammatory Activity of Purified Extracts of Mitragyna speciosa and Cannabis in Combination

Albino Balb/C mice (25-32 g) of both sexes were used. Animals were fasted for 24 h before treatment. The acute inflammatory test was performed using a subplantar carrageenan-induced paw oedema, as described in the literature (Morris 2003). Paw oedema was measured by using a plethysmometer (mod. 7140, Ugo Basile, Italy). Statistical analyses were performed using one-way ANOVA followed by the Newman Keulse test. The topical formulations gel cream o/w, and cream w/o (concentration: 0.5% or 1% alkaloids and 0.5 or 1% cannabinoids) have been applied twice before a subplantar carrageenan injection and once just after. Both concentrations displayed an evident anti-inflammatory activity which reduced, within 4 h and 5 h, the development of the paw induced oedema.

Table 1 shows the synergistic anti-inflammatory effect of a preparation containing Mitragyna speciosa extract and Cannabis extract, compared to preparations containing Mitragyna speciosa or Cannabis extracts alone, for two overall amounts of active principles (0.5% and 1%). The anti-inflammatory effect of the preparations tested is reported as percentage of the anti-inflammatory effect of a similar cream containing betamethasone 1%.

TABLE 1

| | | Anti-inflammatory effect: % |
|---|---|---|
| Purified | Concentration in | vs drug (betamethasone 1% |
| extracts | w/o cream | w/o cream) |
| Mitragyna | 0.5% alkaloids | 44% |
| Mitragyna | 1% alkaloids | 74% |
| Cannabis | 0.5% cannabinoids | 22% |
| Cannabis | 1% cannabinoids | 45% |
| Mitragyna + | 0.25% alkaloids + 0.25% | 56% |
| Cannabis | cannabinoids | |
| Mitragyna + | 0.5% alkaloids + 0.5% | 81% |
| Cannabis | cannabinoids | |

The table clearly shows the improved anti-inflammatory effect of the preparations according to the invention with respect to preparations containing either alkaloids alone or cannabinoids alone, for a same total amount of active principle(s).

B): In vivo evaluation of anti-psoriatic activity of Mitragyna speciosa and Cannabis purified extracts in combination.

Psoriasis is a chronic immune-mediated inflammatory skin disease characterized by keratinocyte hyper-proliferation. For treating mild to moderate psoriatic condition, topical therapy remains the most appropriate option. Corticosteroids are majorly used for topical application to psoriatic skin. Other formulations containing vitamin D3 and its analogues, calcineurin inhibitors, retinoids, tar, dithranol and keratolytic agents such as salicylic acid and urea are also available in different combinations for psoriasis treatment. However, most of these topical therapies have a limited efficacy and may cause various side effects including skin irritation, skin atrophy, skin thinning, pruritus, folliculitis and dryness.

Induction of Psoriasis. Albino Balb/C mice (25-32 g) were sensitized by application of 100 μl of 1.5% oxazolone dissolved in a mixture of acetone and olive oil (4:1) to the shaved abdomen. The animals were re-challenged by applying 20 μl of 1% oxazolone on both sides of the ears every 3rd day starting from 7th day after sensitization. Ear thickness was measured using vernier calliper (Mitutoyo, Japan), after 72 h of application of the oxazolone. The development of psoriatic-like skin was evaluated by determining the extent of ear swelling, erythema, plaque development on the skin surface after every 72 h of repeated application of oxazolone. Test agents: Mitragyna speciosa or Cannabis purified extract (concentration: 1% alkaloids or 1% cannabinoids) w/o cream was applied to both sides of the ear 30 min before and 3 h after each application of oxazolone. The results of Mitragyna alkaloids 1% w/o cream-treated group and Cannabis cannabinoids 1% w/o cream-treated group were compared with drug-treated group (drug: betamethasone 1% w/o cream).

Application of Mitragyna alkaloids and Cannabis cannabinoids 1% w/o cream to the ears of Albino mice with oxazolone induced psoriasis showed a reduction in erythema and oedema. Acute and repeated dermal toxicity studies of Mitragyna alkaloids and Cannabis cannabinoids 1% w/o cream did not reveal any adverse events confirming the safety.

The evaluation was repeated, under similar test conditions, with the w/o cream containing 0.5% Mitragyna alkaloids and 0.5% Cannabis cannabinoids.

The present data demonstrated that the topical use of Mitragyna speciosa and Cannabis purified extracts in combination in w/o cream is a safe and effective anti-psoriatic agent when tested in animal models. The efficacy in preclinical studies could further be exploited for the development of potential novel topical anti-psoriatic agent for therapy in humans.

Table 2 reports the results of the evaluation of an overall concentration 1% of active principles (alkaloids and cannabinoids alone, both alkaloids and cannabinoids). Like in Table 1, the anti-psoriatic effect of the preparations tested is reported as percentage of the anti-psoriatic effect of a similar cream containing betamethasone 1%.

TABLE 2

| | | Anti-inflammatory effect % |
|---|---|---|
| | Concentration in | vs drug (betamethasone 1% |
| Purified extracts | w/o cream | w/o cream) |
| Mitragyna | 1% alkaloids | 79% |
| Cannabis | 1% cannabinoids | 55% |
| Mitragyna + | 0.5% alkaloids + 0.5% | 88% |
| Cannabis | cannabinoids | |

Again, the effect of the invention is significantly better than that of compositions containing only either extract.

### Example 6

For inhalation tests two formulations have been prepared:

1. Solution for modified e-cigarette device for aerosol administration: 50% propylene glycol, 30% glycerine, 2:1 propylene glycol/glycerol solution with 0.1-0.5% Mitragyna alkaloids and 0.1-0.5% cannabinoids and distilled water q.s. (used in mice). Measurements of the aerosol droplets showed to be less than 5.0 μm in diameter suggesting that they were sufficiently small to penetrate deeply into the lungs. Inhalation exposure to aerosolized Mitragyna alkaloids and cannabinoids at very low concentrations in mice elicited anti-inflammatory and antinociceptive effects otherwise less effective with each single extract.

2. Pressurized spray preparation: 2:1 propylene glycol/glycerol solution with 0.1-0.5% Mitragyna alkaloids and 0.1-0.5% cannabinoids in the formulation with polysorbate 80, sorbitan laurate, hydroxypropylcellulose, citric acid and sodium citrate, benzalkonium chloride and distilled water q.s. (to be used in human tests).

An in vivo evaluation of the anti-inflammatory and anti-nociceptive activity of inhalatory delivery (aerosol) of purified extracts of Mitragyna speciosa and Cannabis in combination at low concentrations was performed on mice, in similar manner to the evaluation of the anti-inflammatory activity described above.

The habituation of mice was carried out in a transparent cage (22 cm×16 cm×13 cm), that also served as observation chamber. Another Plexiglas cage was turned upside down and placed over the first cage, in order to avoid any leaks of aerosol.

Albino Balb/C mice (25-32 g) of both sexes were used. Animals were fasted for 24 h before treatment. The test was performed using a subplantar carrageenan-induced paw oedema, as described in the literature (Morris 2003). The inhalation exerted anti-inflammatory and anti-nociceptive effects. In particular, it reduced the carrageenan-induced licking/biting behaviour in a manner that was dependent on the volume of nebulized solution used in the device for its release and on the time of exposure to low concentration alkaloids and cannabinoids solution. Statistical analyses were performed using one-way ANOVA followed by the Newman Keulse test. The aerosol formulations (concentration: 0.1-0.5% of alkaloids and 0.1-0.5% of cannabinoids) have been nebulized in the cage for 60 sec just before a subplantar carrageenan injection.

Table 3 reports the results of the evaluation in terms of pain reduction relative to untreated specimen. As before, the evaluation was carried out with an overall concentration 1% of active principles (alkaloids and cannabinoids alone, both alkaloids and cannabinoids).

TABLE 3

| | Concentration in aerosol | |
|---|---|---|
| Purified extracts | solution | 1-10 scale pain reduction* |
| Mitragyna | 1% alkaloids | 7 |
| Cannabis | 1% cannabinoids | 8 |
| Mitragyna + | 0.5% alkaloids + 0.5% | 6 |
| Cannabis | cannabinoids | |
| Untreated | — | 10 |

*Based on counting licking/biting movements in 3 min

A significant improvement relative to the use of a single active principle was achieved also in this case, with a halved concentration of the individual active principles.

It is clear that the above description has been given only by way of non-limiting example and that changes and modifications are possible without departing from the scope of the invention as claimed in the following claims.

### BIBLIOGRAPHY

World Health Organization: Essential medicines and health products: forty-first meeting of the Expert Committee on Drug Dependence, 24 Sep. 2018. https://www.who.int/medicines/access/controlled-substances/en

Challapalli, P. V. N., Stinchcomb, A. L. In vitro experiment optimization for measuring tetrahydrocannabinol skin permeation. Int. J. Pharm., 2002, 241: 329, 33.

Ali A, Akhtar N. The safety and efficacy of 3% Cannabis seeds extract cream for reduction of human cheek skin sebum and erythema content. Pak. J. Pharm. Sci., 2015, 28 (4).1389-1395

Morris C. J. (2003) Carrageenan-Induced Paw Edema in the Rat and Mouse. In: Winyard P. G., Willoughby D. A. (eds) Inflammation Protocols. Methods in Molecular Biology, vol. 225. Humana Press.

1. A cosmetic and pharmaceutical preparation for topical or inhalatory use containing one or more alkaloids of Mitragyna speciosa and one or more cannabinoids of Cannabis indica or Cannabis sativa, wherein the preparation contains at most 20 wt.-%, of said one or more alkaloids and at most 20 wt.-%, of said one or more cannabinoids.

2. The preparation as claimed in claim 1, containing from about 0.00001 wt.-% to about 20 wt.-% of said one or more alkaloids, and from about 0.00001 wt.-% to about 20 wt.-% of one or more cannabinoids, the weight percentages being related to the total amount of the preparation.

3. The preparation as claimed in claim 1, wherein said one or more alkaloids and said one or more cannabinoids are present as purified extracts of Mitragyna speciosa and a Cannabis indica or Cannabis sativa, respectively, or are present as isolated alkaloids and cannabinoids, respectively.

4. The preparation as claimed in claim 1, wherein said one or more alkaloids are selected from: speciociliatine (SC), 3-isopaynantheine (IP), speciogynine (SG), paynantheine (P) and mitragynine (MG); and said one or more cannabinoids are selected from Δ(9)-tetrahydrocannabinol (THC), cannabidiol (CBD), tetrahydro-cannabinolic acid (THCA) and cannabidiolic acid (CBDA).

5. The preparation as claimed in claim 1, further containing an enhancer or ethosomal carrier for improving transdermal permeation of more lipophilic alkaloids and cannabinoids.

6. The preparation as claimed in claim 1, having a dosage form selected from ointment, O/W-emulsion, W/O-emulsion, dispersion, lotion, gel, mousse, tincture, spray, aerosol, foam, powder, tape and patch.

7. A method of preventing and/or treating skin and mucosal disorders and/or conditions or disorders and/or conditions of the respiratory tract, comprising the step of using the preparation according to claim 1.

8. The method as claimed in claim 7, wherein said skin disorders and/or conditions include ageing and redness, inflammation, acne, atopic dermatitis, psoriasis, couperose, dandruff, scalp disorders, excessive sweating (hyperhidrosis), pruritus and sub-cutaneous pain.

9. The method as claimed in claim 7, wherein said mucosal disorders and/or conditions include lip disorders, rhagades and haemorrhoids.

10. The method as claimed in claim 7, wherein said disorders and/or conditions of the respiratory tract include sinusitis, adenoiditis, laryngitis, nasopharyngitis, chronic rhinitis, chronic pharyngitis and tracheitis, bronchiolitis and bronchitis.

11. The method as claimed in claim 7, wherein said preparation is used in a dosage form for topical or inhalatory administration.

12. The method as claimed in claim 11, wherein said preparation is used in a dosage form for topical administration in combination with a dosage form for systemic administration.

13. A method of treating a human being suffering from skin and mucosal disorders and/or diseases and/or conditions, comprising topical and/or inhalatory administration of the preparation as claimed in claim 1.

14. A method according to claim 13, including topical or inhalatory administration of the preparation combined with a systemic administration.

15. The preparation according to claim 2, containing from about 0.0001 wt.-% to about 10 wt.-% of said one or more alkaloids.

16. The preparation according to claim 2, containing from about 0.0001 wt.-% to about 10 wt.-% of one or more cannabinoids.

17. The preparation according to claim 2, containing from about 0.1 wt.-% to about 5 wt.-% of said one or more alkaloids.

18. The preparation according to claim 2, containing from about 0.1 wt.-% to about 5 wt.-% of one or more cannabinoids.

19. The preparation according to claim 1, further containing an enhancer or ethosomal carrier for improving transdermal permeation of mitragynine (MG) and tetrahydrocannabinol (THC).
